(19) United States
(12) Patent Application Publication
(10) Pub. No.: US 2018/0161087 A1
(43) Pub. Date: Jun. 14, 2018

(54) MATTER MANIPULATOR WITH CONDUCTIVE COATING

(71) Applicant: GI SCIENTIFIC LLC, Arlington, VA (US)

(72) Inventor: Scott MILLER, Arlington, VA (US)

(21) Appl. No.: 15/579,046

(22) PCT Filed: Jun. 2, 2016
(86) PCT No.: PCT/US2016/035593

§ 371 Date: Dec. 1, 2017

Related U.S. Application Data
(60) Provisional application No. 62/170,010, filed on Jun. 2, 2015.

(51) Int. Cl. A61B 18/14; A61B 18/12

(52) U.S. Cl. CPC A61B 18/14; A61B 18/1206; A61B 2018/00595; A61B 2018/1405; A61B 2018/126

(57) ABSTRACT

A device including a tissue manipulator, a conductive coating and at least one connector area. For example, the tissue manipulator may be scissors, clip appliers or clips, staplers and staple or a vessel sealing device. The conductive coating may be applied to the clip, staple or jaws of the scissors or sealing device. Electrical energy can be supplied through areas of contact (connector areas)—such as between an anvil and a pusher of the stapler and the conductive coating on the staple. The conductive coating can be energized along with mechanical application of the manipulator to transform and facilitate attachment of tissue layers.

### CROSS-REFERENCE TO RELATED APPLICATIONS

This application claims benefit of U.S. Provisional Application No. 62/170,010, filed Jun. 2, 2015, the entirety of which is incorporated herein by reference.

### BACKGROUND

Minimally and less invasive surgery, and interventional treatments, of patients are generally safer, faster, and less traumatic to the patient. These procedures therefore involve less inflammation, post-operative pain, infection risk, and reduced healing time compared to more invasive forms of surgery, including general and open surgery.

Similarly, in non-medical applications, less invasive inspection and repair of remote areas and defects in non-medical settings, whether involving a sewer trunk line, a hydraulic line, an oil pipeline, a gas line or other non-medical areas in which inspection and/or repair can be obtained with less disruption and intrusion, is generally superior to opening up the area more invasively to inspect and repair.

In medical applications, less invasive approaches usually involve either direct (or remote) visualization with instruments used for diagnosis and for treatment and manipulation. Direct visualization applications include surgery using a small incision (called a mini-thoracotomy) and direct visualization of an open, general surgical site. Alternatively, one or more forms of remote visualization may be used, such as an inspection of the colon using a flexible colonoscope or visualization of a surgical site using a laparoscope, or imaging of a vessel or a lumen using contrast media and fluoroscopy while navigating inside the vessel using guidewires and catheters.

In non-medical applications, direct visualization may be achieved through the use of small ports. For example, by drilling a hole into a pipeline to inspect the line at a specific point. Another example is the use of a borescope to remotely navigate and advance through the pipeline to visualize the area of inspection for possible remote repair.

Despite the benefits with these approaches, there exists a need to improve the overall visualization and manipulation of tissue and other matter through the addition of more therapeutic and repair capabilities for use in both medical and non-medical applications. Matter manipulators for open procedures (medical and non-medical) may also benefit from additional improvements.

### SUMMARY

Implementations of the present disclosure overcome the problems of the prior art by providing a device with at least a matter manipulator, a conductive coating and a terminal. The conductive material is disposed on at least a portion of the manipulator. The manipulator, for example, may be a staple, a knife, a wire, a snare, a grasper or a dissection element, sutures, mesh and other implantable devices (including a spinal cage, stents, heart valves, defibrillators and pacemakers, knee and hip replacements, and other implantable devices). The terminal is capable of providing energy (such as electrical energy) to the conductive material. In one aspect, the conductive material is an optically transparent material. Also, the conductive material may conduct electrical energy. Advantageously, the device allows manipulation of tissue or other matter concurrent with the application of energy via the conductive coating.

These and other features and advantages of the implementations of the present disclosure will become more readily apparent to those skilled in the art upon consideration of the following detailed description and accompanying drawings, which describe embodiments of the present disclosure.

### BRIEF DESCRIPTION OF THE DRAWINGS

FIG. 1 shows a schematic of a device of one implementation of the present invention including a surgical staple with a conductive coating and a power source;

FIG. 2 shows a schematic of a series of surgical staples with a conductive coating, such as the staples of FIG. 1 in series;

FIG. 3 shows a schematic of a surgical stapler with a conductive coating and a power source;

FIG. 4 shows a schematic of a bi-polar snare with a conductive coating and a power source;

FIG. 5 shows a schematic of a surgical suture with a needle, either or both of which may have with a conductive coating, and a power source;

FIG. 6 shows a schematic of a surgical mesh with a conductive coating and an organic power source and an external power source;

FIG. 7 shows a schematic of a stent with a conductive coating, a delivery catheter and a power source;

FIG. 8 shows a schematic of a heart valve repair ring with a conductive coating, an organic power source and an external power source;

FIG. 9 shows a schematic of a surgical knife with a conductive coating and a power source;

FIG. 10 shows a schematic of an articulating and telescoping matter manipulator with graspers at the distal end wherein the graspers have a conductive coating;

FIG. 11 shows a schematic of a spinal cage with a conductive coating, an organic power source and an external power source; and

FIG. 12 shows a schematic diagram of a device of yet another implementation of the present invention.

### DETAILED DESCRIPTION

Implementations of the present disclosure now will be described more fully hereinafter. Indeed, these implementations can be embodied in many different forms and should not be construed as limited to the implementations set forth herein; rather, these implementations are provided so that this disclosure will satisfy applicable legal requirements. As used in the specification, and in the appended claims, the singular forms “a”, “an”, “the”, include plural referents unless the context clearly dictates otherwise. The term “comprising” and variations thereof as used herein is used synonymously with the term “including” and variations thereof and are open, non-limiting terms.

The inventors have observed that despite the many benefits associated with using devices to address matter less invasively, including remote visualization, in a medical context or to inspect and fix conditions in non-medical applications, as well as in open procedures such as general surgery and non-medical applications, there still are significant issues with these technologies where improvement is needed. Instruments and other elements to modify, manipulate and repair matter and to provide other therapy typically have single or limited capabilities, including a lack of ability to effectively delivery energy, while performing other beneficial tasks.

The present invention in some implementations, includes a matter manipulator with one or more coatings on the manipulator, including a conductive coating, which allows energy to be applied to the matter in contact or proximity to the manipulator to alter or affect matter. Implementations of the invention have the benefit of enabling matter manipulation and energy delivery in the same device. Examples include providing energy applications to devices used for grasping, dissecting, snaring, manipulating, debriding, sealing, measuring, assessing, navigating and closing tissue and other matter. Further, the conductive coating has the benefit of transmitting energy on or across the coating, limiting the effect of the energy on the manipulator if desired, including limiting self-heating and unintended collateral effects from the energy, including thermal spread.

Examples of applications of the matter manipulator with conductive coating include applying the matter manipulator to seal vessels or as a surgical knife with reduced self heating of the device and sticking of the vessels, applying the matter manipulator to snares to provide uniform bipolar application of energy to excise polyps in the gastrointestinal tract, applying the matter manipulator to surgical graspers and stents to deliver energy to tissue and other matter, among others.

Embodiments of the device include matter manipulators with conductive coatings that also have hydrophobic and superhydrophobic water contact angles, which prevent matter from sticking or adhering to the matter manipulator, including when energy is applied to the matter through the conductive coating. This allows the energy to be delivered to the matter without unintended manipulation of the matter due to sticking, charring or other unintended adhering of the matter to the manipulator. In addition, it allows the energy to be delivered across the coating, rather than through the manipulator, reducing the level of self-heating and collateral thermal spread. This has performance advantages with a variety of applications, such as surgical knifes, surgical staples, clips, clamps and other securement devices, the application of glues and other adhesives that are activated or cured by energy, graspers that can now be energized without thermal spread, colorectal snares that can be energized without thermal spread and without gaps in the application of energy to tissue, and other applications that benefit from the application of energy and a conductive coating to limit matter adherence. This variant of the manipulator may also include long-term implantable devices where the application of energy through the coating lowers the incidence of bleeding with the implant of the device and can prepare the surrounding matter for accepting the device, while the hydrophobic or superhydrophobic water contact angle prevents encapsulation and tissue in-growth, reducing certain infection risk and preserving the option to remove the implant if needed.

Examples of this application include, among others, pacing leads and pacemakers, implantable defribrilators, removable sutures and staples, certain types of stents (such as biliary stents used to open blocked ducts, which may later be removed), endographs, breast implants, penile implants, neurostimulation devices for treating neurogenerative diseases (such as Parkinson's disease and depression), stimulation devices for treating arthritis and for pain management, knee and hip implants and other implantable devices where the application of energy and limited or restricted tissue in-growth is beneficial. Further, these devices may be configured so that the conductive energy comes from an external source, such as an energy generator, from an energy source in the device, such as a battery, or from an organic source, such as a connection to an energy generating element in the implant object, such as a nerve or other electrical impulse element in the patient.

Embodiments of the matter manipulator also include variations of the conductive coating that has hydrophilic water contact angles, which encourages the retention of water and the adherence or in-growth of tissue and other matter to the manipulator, including when energy is applied to the matter through the conductive coating. This allows the energy to be delivered to the matter while encouraging adherence of the matter to the coating to create performance benefits through the combination of the application of energy and the hydrophilic coating. Examples of these benefits include improving the level of tissue ingrowth in medical applications where the manipulation of matter, application of energy and tissue in-growth or adherence is beneficial, such as with bio-absorbable and non-absorbable sutures, surgical meshes for various repairs (including hernia, pelvic floor, incontinence, breast reconstruction and other reconstructive surgeries), coils for use in lumens (including, for example, the vascular system, lungs, uterus and neurovascular system), certain stents (such as a stent or similar implant used to close a fallopian tube), heart valves, heart valve rings, dental implants and gum grafts, among others.

A matter manipulator with a conductive coating would be beneficial also in nonmedical applications, including applications that would benefit further from having a conductive coating with various water contact angles, including a matter manipulator with a hydrophobic or a superhydrophobic water contact angle, or a hydrophilic water contact angle. Examples of applications include, for example, to apply energy to remove or clear debris in a pipeline or sewer line, to cure a glue or adhesive to repair a defect, to weld together weldable materials at a unique juncture in which the manipulator is configured to fit the juncture, to deliver energy a distance from the user through a relatively long element with the matter manipulator on the end of the device (or arrayed across the device consistently or intermittently), including unitary and connecting and/or telescoping devices, and others forms of extending or navigating the manipulator to matter.

The matter manipulator with conductive coating can be a hand instrument (whether rigid or articulating or a combination thereof), a remote navigation instrument (such as a wire, a catheter, a scope or other similar device), one or more robotic arms (including part of a robotic system), an implantable device in a medical or non-medical application, a part of a production line where the manipulation of matter and the application of energy with various water contact angles is advantageous, as part of a manufacturing mold, and any other application where the manipulation of matter and the application of energy with various water contact angles is advantageous or beneficial.

The matter manipulator may also be used to deliver energy in a variety of forms, including electrical energy delivered as direct current, alternative current, high-voltage pulsed current, low-intensity direct current, a pulsed electromagnetic field, frequency rhythmic electrical modulation, and other forms of electrical energy with a therapeutic or beneficial effect on tissue or other matter. Examples of the medical benefits from the application of these various energy forms include improved fracture repair, reduced pain (from transcutaneous nerve stimulation and other electrical stimulation approaches), reduced bacterial load, cell modification or proliferation, improved perfusion, accelerated wound healing and targeted tissue or matter modification.

In embodiments, the manipulator may be articulating, flexible or rigid or a combination of these features.

In embodiments, the matter manipulator includes feedback elements to allow the operator to deliver precise amounts of energy to matter, including pressure sensors, thermistors, thermocouplers, ultrasound and other forms of imaging, and other feedback and location and navigation elements to improve the level of precision and accuracy with delivering energy to target matter, as well as to improve the navigation to matter targets.

Generally, as an additional example, the inventors have found in less invasive surgery that instruments need to be advanced, retracted and exchanged through incisions, ports, working channels or other points of access. This approach means the correct instrument is not always readily available when needed. For example, when performing a laparoscopic surgery case, a blood vessel may be cut and a bleed occur, while the physician is engaged in fine dissection of tissue to access a treatment point. The physician may not have a cautery or vessel sealing instrument in one of the ports used to advance and retract instruments in the patient for treatment. When this occurs, the bleed will continue while the physician retracts one of the instruments and inserts a cautery device or a vessel sealing device (called a device exchange) to try and then find the bleed and stop it. Due to the time it takes to complete the device exchange, the bleeding area may become filled with blood, obscuring the location of the bleed. Further, during this time, the scope may become covered with blood, debris or other fluid, or may fog causing additional issues that complicate finding and treating the bleed.

A matter manipulator that is able to manipulate matter and deliver energy rapidly to address a bleed, for example, without engaging in a device exchange is valuable. In some instances, the matter manipulator could be deployed with or using a scope with an optical coupler with (or without) a conductive coating. But, in other instances, the physician may want to keep the scope distant from the bleed and use a different instrument at a different angle to address the bleed, which is where a separate matter manipulator with a conductive coating would be of special value, overcoming current practice limitations.

Also, a certain substrate stability is desirable when used with energy applications to minimize the impact of the substrate on the conductive materials and the impact of the energy delivery on the substrate. The material for the substrate can be any material that provides the level of adherence for the conductive coating and its targeted energy application. These materials can include, for example, polycarbonate, acrylic, polystyrene, cyclic olefin copolymer, cyclic olefin polymer, polyethermide, quartz, glass, aluminum, bioabsorbable materials, nitenol, steel, silicone, other elastic materials, other elastomeric materials, other metals, ceramic materials, epoxies, grapheme, and any other material suitable for the adherence of the conductive coating in the given application, taking into account coating adherence, temperature performance, biocompatibility when applicable, durability, ease of manufacture, and other factors.

For example, polycarbonate materials are a well-suited material for certain applications, including those where an optically clear substrate may be desired, because of polycarbonate's index of refraction and performance across various temperature ranges. These materials provide appropriate temperature performance, including insulation properties and relatively low levels of thermal expansion when used with the application of various forms of energy. Further, some of these materials provide an additional combination of a relatively low index of refraction, and high light transmission for applications where these additional properties are beneficial.

The device may also have one or more other coatings, including another conductive coating, a dielectric coating, and other coatings (including radiopaque coatings or markers) or conductive or insulation material to facilitate the effective delivery of energy to matter.

In other embodiments, a device using more than one material may connect the materials through glue or other chemical bond, molding the materials together, over-molding one material on the other material, placing a mechanical connector between the materials, or over the materials, or a combination thereof. Connections may also be made by coating one material onto another, screwing one material on to the other, inserting a wire, or other ways of connecting one material to another wherein at least one of the materials is a substrate for a conductive coating.

Embodiments of the matter manipulator include at least some transparent structure for the matter manipulator that, when combined with a transparent conductive material, allow some improved visibility of the matter being manipulated. The term “transparent” as used herein is not always limited to optically transparent. Instead transparent may include the ability to or characteristic of passage of energy waves, including infrared and/or ultraviolet rays. Transparent also need not be limited to perfectly transparent and instead could refer to some ability to facilitate or allow passage of light rays (e.g., translucent).

Alternatively, the manipulator may not be formed of any transparent material and in embodiments can be made from one or more non-transparent materials suitable for the particular manipulator application. In embodiments, for certain applications, the manipulator may serve as a support and applicator for the conductive material with either limited or no ability to improve visualization.

It should be noted that the manipulator may be a single device, a device delivering other devices (like a staple) or it may attach to another device (including a navigation instrument) via an attachment section. The attachment section may include other structure to facilitate attachment and/or may be secured by welding, adhesive, screwing, mechanical connectors, interference between one or more materials and the other device (such as an optical imaging device), or such other form of connection between the device and another device. Also, the attachment section need not have a particular shape (such as a cylinder) but instead can be formed to match the distal end shapes of various optical imaging devices or remote visualization devices or navigation devices. Or, the attachment section may be shaped to facilitate other functions of the device, including shaping the sides and distal end to conform to and manipulate tissue and matter more effectively. Shapes and materials may be selected to make the device less traumatic when contacting tissue and other matter.

In some embodiments, the conductive material may be in the form of a layer, strip, particle, nanoparticle or other shape applied in some discrete, continuous or intermittent pattern and in various combinations thereof. Variations in the shapes or patterns of application of the conductive material are possible within the capabilities of adding one material to another by adhering or combining the coating and other materials to achieve a desired result.

The conductive material can comprise a transparent conductive oxide (TCO), a conductive metal such as platinum, a polymer, or an organic semiconductor or such other materials able to conduct or transmit energy across the device. The term “layer” refers to at least some area of the conductive material having a relatively uniform thickness and/or the method of application of the conductive material. For instance, the conductive material may be formed or applied through dipping, deposition coating, spraying, sputtering, ultrasonic application, brushing, painting, direct ion beam deposition, pulsed laser ablation, filtered cathodic arc deposition, ion beam conversion of condensed precursor, magnetron sputtering, radiofrequency plasma-activated chemical vapor deposition, or such other application of the conductive material able to form a layer or other pattern on the intended substrate. In some embodiments, the conductive material may be of a uniform material thickness. In other embodiments, the conductive material may have a varying thickness. No part of the conductive material need be of an exact thickness—it could vary continuously throughout. Instead, material thickness can be varied depending on the intended electrode function, such as the target level of resistance (and its variations) across the coating for the specific application.

Further, the conductive material may be applied to form particular shapes (other than a layer) meant to apply energy in different patterns and densities to matter. Also, the conductive material may be applied in a non-layer like manner, such as by being formed in a mould and then adhered, welded or otherwise attached to the matter manipulator. Again, the shape of the conductive material instead may correspond to the desired pattern of energy application by the conductive material, including a specific electrode design involving the conductive material and connectors to the conductive material.

In embodiments, the conductive material may be applied in a pattern (strips, stripes, dimples, voids, undulations, curves, circles, semicircles), irregular, and such other approaches to create an electrode for an intended result applying energy with the device.

Additional Exemplary Matter Manipulators with Conductive Coatings

In other implementations, matter (such as tissue) manipulators may include the conductive coating to energize the manipulator. For example, a device might include a tissue manipulator, a conductive coating disposed on the tissue manipulator and a connector that is configured to supply energy to the conductive coating. The term “connector” as used herein should be construed broadly to mean any structure that enables electrical or other energy communication to the conductive coating. The term “connector” can refer to a permanent connection (solder, gluing, twisted wires, a conductive path with a conductive coating) or exchangeable connectors, like a plug and harness assembly, or other way of transmitting energy from a power source towards the conductive coating. It need not be a physical connection all the way through to the coating. It could, for example, connect via electromagnetic field—such as by inductance. The term “connector” may also include structure and/or function that allows, mediates, enhances or otherwise facilitates a connection. A particular type of connector is a terminal that may be, for example, an area of conductive material provided for or capable of electrical coupling with a power source. A terminal, for example, may be a conductive metal layer deposed on a surface and shaped for contact with an end of a wire on an energy supply catheter.

A “connector area” is an area where the connector can be attached, mounted, coated, inserted, contacted, connected, glued, affixed, adhered, layers, overlapped or can otherwise communicate energy to the conductive coating.

The term “matter manipulator” as used herein refers to any device for applying a force to matter—such as for snaring, capturing, cutting, fastening or otherwise moving or affecting matter, preferably through restricted openings. In particular, the matter manipulator may be one of a range of tissue manipulators operated through small ports in patient tissues and anatomy during minimally invasive surgery. Non-tissue matter could also be manipulated, such as matter trapped in piping systems or in constricted areas.

In one example, the conductive coating can be applied to an endoscopic stapler to energize the staple as it is applied to tissue. Energizing the staple can further close the two (or more) tissue planes bound together by the staple. For example, energy may applied while the staple extends through the tissue planes to deliver energy across and into the tissue, while minimizing the heating of the staple. The energy from the conductive coating then affects the tissue planes to cauterize or cross-link them for additional security beyond just the mechanical force of the staple itself.

The coating may be employed to alter tissue in a number of ways. Tissue alteration may, for example, include ablation, cauterizing, shaping, sealing, dissecting, resecting, cutting and coagulating tissue.

U.S. Pat. Nos. 5,040,715; 5,413,268 and 5,476,206 entitled APPARATUS AND METHOD FOR PLACING STAPLES IN LAPARASCOPIC OR ENDOSCOPE PROCEDURES, which are hereby incorporated herein by reference in their entirety, disclose staplers which can be modified for use with the conductive coating. FIG. 13 of the '206 patent, for example, shows a side view of a stapler cartridge assembly 137. An anvil member 136 includes an anvil plate 136A, a tissue contacting surface 136B and staple forming depressions 136D. The stapler cartridge assembly also includes pushers 139 that engage staples 138 as the pushers are sequentially engaged by cam bars 131.

In the exemplary configuration of the '206 patent, the staples themselves may be coated partially or fully by the conductive material as described above. The staples may be, for example, coated in a dipping process before being loaded into the cartridge 137. Also, the anvil member 136 and/or the pushers 139 and/or cam bars 131 may be connected to an electrical power supply. (The electrical power supply could be run from the proximal end of the stapler assembly via wires through the device to connect to the anvil member 136 and cam bars 131.)

As shown in FIG. 13 of the '206 patent, one staple 138 has been pressed through the tissue layers 201 and 202 and into the adjacent forming depression 136D. The forming depression bends the arms of the staple back on itself to mechanically attach the two tissue layers together. At this moment, the staple coating can be energized by ending electricity through the anvil 136 and the cam bar 131 and adjacent pusher 139. The electrical energy causes the conductive coating to deliver energy to the tissue and facilitate sealing the tissue layers together, e.g., such as by sealing, cauterizing or reformulating the tissues. Advantageously, the additional sealing of the tissue layers is implemented in a minimally invasive setting—where access and delivery of energy is more challenging.

The conductive coating is not limited to the staplers disclosed in the '206 patent could be applied to other staplers (or fasteners), such as those disclosed in U.S. Pat. Nos. 5,040,715; 5,137,198; 5,326,013; 5,657,921; 5,662,258; 6,131,789; 6,981,628 and 6,988,650 which are hereby incorporated by reference in their entirety. U.S. Pat. No. 6,981,628 for example discloses a lateral-articulating stapler. As a further example, electrical wires supplying power to the conductive material coating could bend and articulate with the stapler shown in the '628 patent.

In another example, the conductive coating may be applied to staples for end-to-end anastomosis of vessels. U.S. Pat. No. 5,104,025, which is hereby incorporated herein by reference in its entirety, discloses a stapler wherein the staples are held circumferentially around the head of a trocar and then pressed into shape against a circular anvil. Similar to the '206 patent above, the staples of the '025 patent may be coated in the conductive material and the stapler equipped with energy supplying wires connected through the anvil and pushers to the coating on the staples. The conductive coating disclosed herein could be applied to other anastomosis surgical stapling instruments, such as U.S. Pat. No. 5,205,459 which is hereby incorporated herein in its entirety by reference.

Other matter manipulators for the conductive coatings include energizing surgical knives, vessel sealing devices, clip appliers or combinations of such devices. U.S. Pat. No. 6,988,650, for example, is a combination curved stapler and cutter wherein coatings could be used in two places and have energy supplied by one or more connectors. The coating can be applied to the surgical knife to cauterize the tissue as it is cut and stapled, and the staple also can be coated so the staple can be energized to limit bleeding and accelerate healing and lower infection risk along the staple line. The coating can also be applied to the vessel sealing device or clip to facilitate sealing or closing of the tissue.

U.S. Pat. No. 8,915,931, which is hereby incorporated herein in its entirety by reference, discloses a surgical clip applier. The '931 patent discloses in FIGS. 1A and 1b a clip 10 [using original reference numbers from the '931 patent] that includes a cutting element 38 at one end to incise tissue as it is clipped. In use, after the arms 14, 16 have been distracted relative to each other, the clip 10 is advanced over a tissue such that the cutting blade 38 of the clip 10 incises the tissue. Once the tissue has been incised to a certain length, the arms 14, 16 can be released and the clip 10 can return to its biased closed state to ligate the incised tissue. This clip 10, including the cutting blade 38, may have applied thereto a conductive coating as described above for transforming the tissue before, during or after application of the clip.

FIGS. 4A-4E of the '931 patent also disclose a clip applier device 100. Jaws 112 on the distal end of the device hold the clip 10 and may be connected to a power source for energizing the clip. The jaws themselves may also be coated and energized for tissue transformation, such as cauterization when clamping tissue between the jaws.

The conductive coating could also be applied to a LIGASURE-like instrument for electrosurgical sealing of blood vessels. The LIGASURE devices use bipolar electrical energy to electrothermally seal blood vessels. Although effective, the LIGASURE must guard against excessive energy application—usually via monitoring the impedance rise of the circuit. Use of the intervening conductive coating could reduce the incidence (or complexity of controlling) over-application of energy and related thermal spread and potential injury to surrounding tissue.

U.S. Pat. No. 7,819,872 discloses a flexible endoscopic catheter with ligasure device. The jaws of the device can be coated with the conductive coating and the existing electrical power supply modified to provide connectors for the conductive coating. Also, the device includes a snare which could similarly be coated with the conductive coating and then energized for tissue transformation.

The matter manipulator may include a tissue closure or repair device. For example, tissue closure or repair devices include devices for closing one or more planes of tissue together. For example, tissue closure or repair devices may include as sutures, staples, fasteners, clips, clamps, anastomosis devices, and glues that can be activated with energy.

The matter manipulator may include a tissue support such as a mesh (or other biocompatible material) for a hernia, vaginal repair, spine and other orthopedic procedures, uterine or other tissue repairs.

The matter manipulator may also include various implants, such as spinal cages, stents, clips (such as, for example, vascular, heart valve or meniscal clips) or various anchoring devices.

Bioabsorbable materials may be employed for the matter manipulators, such as in sutures, staples, clips, anchors, meshes and stents. Synthetic or biologic materials materials may also be employed for matter manipulators, such as synthetic meshes.

Tissue manipulators may include energy probes such as bipoloar energy probes. The coating on these probes and other matter manipulators, in addition to being conductive, may also be hydrophilic, hydrophobic or even super-hydrophobic. Advantages of controlling hydrophilicity/hydrophobicity include controlling the water contact angle. Water contact angles for example can be 90, 140 or even 170 degrees. Controlling hydrophobicity or hydrophilicity will allow facilitation of tissue ingrowth for repair devices or avoidance of tissue sticking or attachment on manipulators. Prior art devices have employed hydrophobic or hydrophilic coatings, but not as part of the energizing material or component. For example, jaws of a LIGASURE may be energized but not the hydrophobic coating itself.

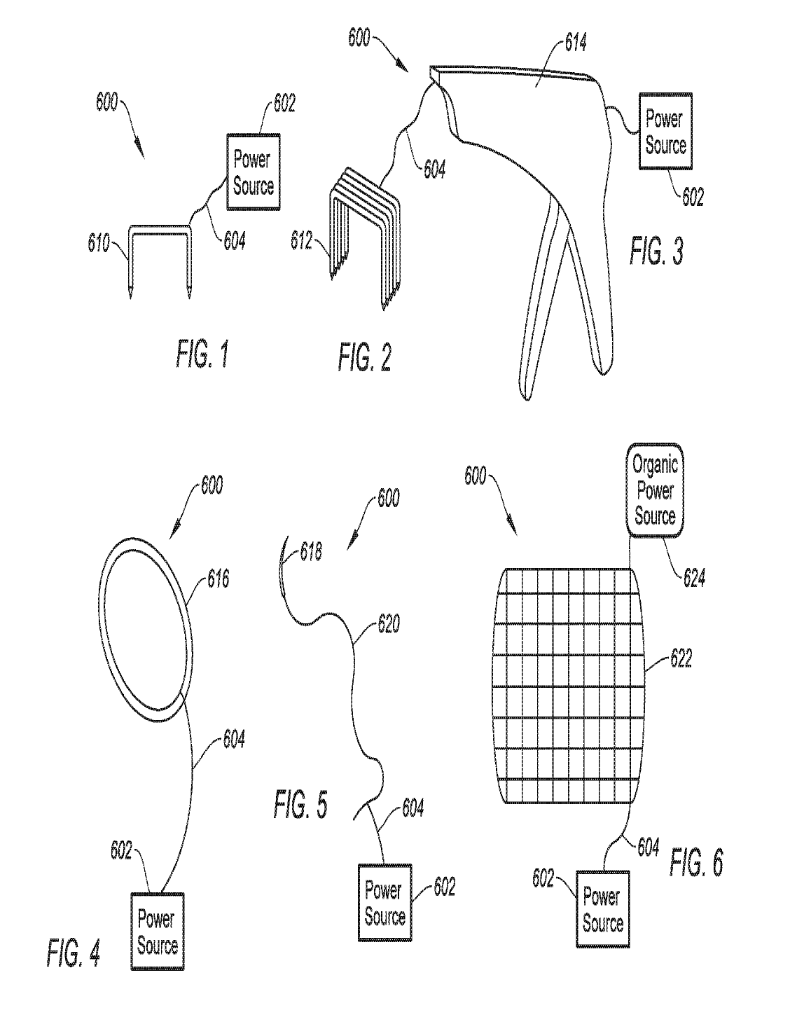

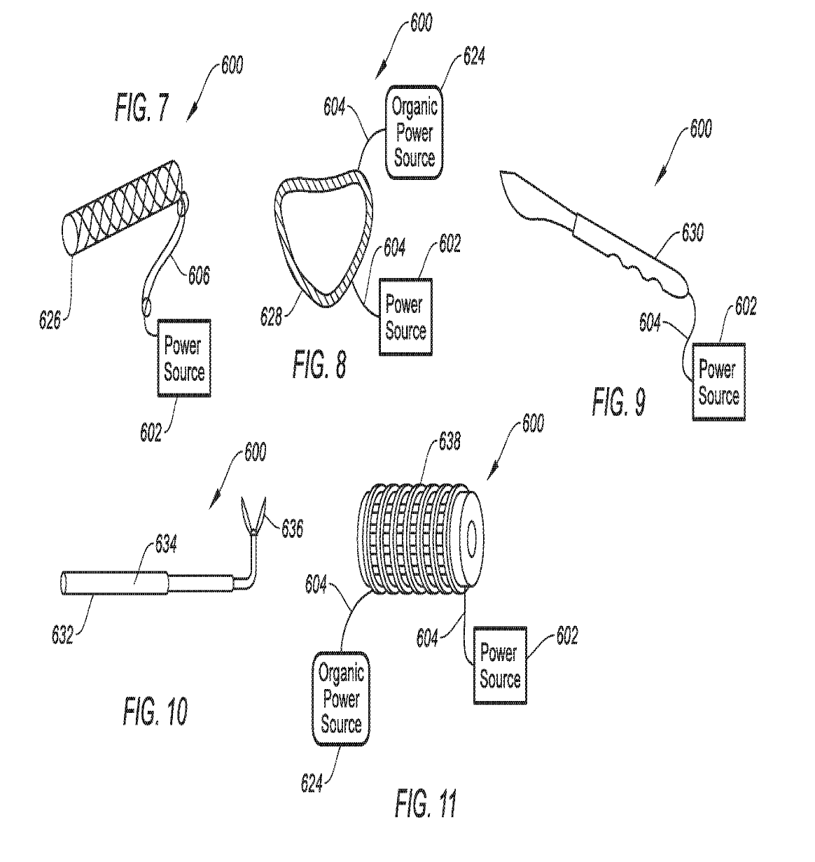

Matter manipulators may also include a guide wire or catheter used for navigation and manipulation. These may also be used within or deployed through a lumen. FIGS. 1-11 show examples of devices 600 with matter manipulators with conductive coatings applied to or near their matter contacting surfaces. FIG. 1 shows a staple 610 connected to a power source 602, such as by wires 604. FIGS. 2 and 3 show a row of staples 612 connected through a stapler 614 to the power source 602. FIG. 4 shows a bipolar snare 616 connected to the power source 602. FIG. 5 shows a needle 618 connected through a surgical suture 620 to the power source 602. FIG. 6 shows a surgical mesh 622 connected to an organic power source 624 and the power source 602. FIG. 7 shows a stent 626 connected through a power delivery catheter 606 to power source 602. FIG. 8 shows a heart valve ring 628 connected to the power source 602 and organic power source 624. FIG. 9 shows a surgical knife 630 connected to power source 602.

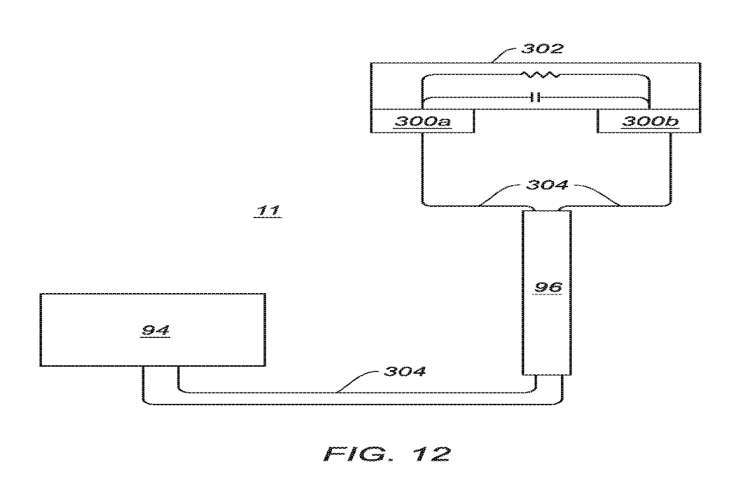

FIG. 10 shows an articulating and telescoping matter manipulator 632 including a telescoping shaft 634 having a bendable end and a pair of graspers 636 at the distal-most free end. The telescoping matter manipulator 632 may, for example, be powered through wired connection to a power source or use of the power delivery catheter 606. FIG. 11 shows a spinal cage 638 connected to power source 602 and organic power source 624.

The telescoping shaft 634 may include a channel for transmitting fluid, air or other matter. This channel can be used to transmit fluid, including water or saline to irrigate tissue or to rinse debris from the field of view or to clean the outer surface of the manipulator, or to transmit drugs and other chemicals, and other matter, such as air, CO₂, argon gas and other matter to effect targeted tissue or other matter. An opening may extend through the conductive material applied to the graspers 636 for aspiration of the external environment as well as applying positive pressure to an instrument receptacle when an instrument is deployed externally.

These (and other) devices may include one or more connectors to provide energy to the conductive material. The connectors, in this implementation, include a first positive terminal and a second negative terminal. Electrical current flows from the positive terminal, through the conductive material (energizing the conductive material) and out through the negative terminal.

The terminals can themselves be comprised of inert electrodes such as graphite (carbon), platinum, gold, and rhodium. Additionally the terminal may comprise copper, zinc, lead, and silver, or aluminum, or the conductive material or any other material known to one skilled in the art to be appropriate for transmitting energy. The wires or other power transmitters connect the electrode to the power source 602. For example, the power delivery catheter 606, as shown in FIG. 7, may include wires 604 embedded within a sheath or extending along a lumen of the delivery catheter.

The wires may also be delivered in another alternative manner, including inductive transmission of current to the device or to a battery embedded in the device. Power may also be supplied by current from a battery, a catheter, a cable, radio waves or other power transmission devices or methods capable of extending a distance to a terminal or connector.

As shown schematically in FIG. 12, a conductive material 302 (used on a matter manipulator) is a resistor and/or capacitor attached via terminals 300a and 300b and a connector 304 and a cable 96 to a power source 94. The connector 304 may extend through (for example) an endoscope sheath 76 and into the cable 96 attached to the endoscope's proximal end. Those connectors may connect to the power source 94 that may, for example, be one or more forms of energy for the alteration of tissue or other matter, including monopolar energy, bipolar energy, argon gas energy, microwave, coblation energy, plasma energy, cryo energy, thermal energy, ultrasound, focused ultrasound or other forms of energy, including the generation and transmission of multiple energy forms which can be transmitted across or through a conductive coating to alter tissue or matter.

The conductive material 302 of the various implementations of the device 11 may be used to deliver many energy types and employed in many medical and non-medical applications. Examples of such energy types and applications are provided elsewhere herein for illustrative purposes and should not be considered limiting.

There are many ways to deliver energy to the terminals 300a, 300b and the conductive material 302. The cable 96 can deliver power to the conductive material by way of the terminals 300a, 300b. The cable can access the terminals by, for example, being wrapped around the outside of a scope. Or, the cable 96 or connector 304 can be attached to an energy delivery catheter that is passed down the working channel of the scope and docks with the terminals. At its distal end, the energy delivery catheter may be connected to an electrical terminal in the working channel of the matter manipulator. The connectors may be comprised of flex circuits, one or more coatings, wires, conductive springs, inductive material for receiving and transmitting power, cables, or such other approaches for transmitting power from a power source toward a deliver point.

The terminal or terminals 300 may be any device (including radio waves, induction or other wireless connection) that delivers energy of some kind to the conductive material 302. The conductive material itself, in the case of wireless excitation or extension of the conductive material into a shape for mating or communicating with an energy generator (or other power source) for example, may form or include the terminals 300.

Insulating material may extend from the surface supporting the layer of conductive material and be of the same, or lesser, or greater thickness than the layer of conductive material 302. Advantageously, the insulating material may prevent a disruption of the conductivity of the conductive material 302, such as by a metal instrument causing a short to an electrically energized conductive material layer. Or, the insulating material may just be a more elastic, physical guard against damage by the matter manipulator.

In other implementations, the matter manipulator is attached to the end of a catheter (such as a scope) and has a frusto-conical shape with the broader base extending distally. In this implementation, the conductive material 302 is relatively flat and can be easily applied to a relatively flat tissue surface. Also, the conductive material 302 may extend in a layer around an opening that is surrounded by an insulating material. This can insulate against a short or damage to the conductive material by other manipulators passing through the opening—such as biopsy forceps. Also, the electrodes 300a and 300b may extend down the angled sides of the frusto-conical shape and may or may not be partially or fully insulated.

In another implementation, the electrical energy generator can comprise a signal generator such as: a function generator, an RF signal generator, a microwave signal generator, a pitch generator, an arbitrary waveform generator, a digital pattern generator or a frequency generator. An existing electrosurgical generator may be used with the advantage that it meets standards necessary for medical use. These generators may provide power to electronic devices that generate repeating or non-repeating electronic signals (in either the analog or digital domains). RF signal generators can range from a few kHz to 6 GHz. Microwave signal generators can cover a much wider frequency range, from less than 1 MHz to at least 20 GHz. Some models go as high as 70 GHz with a direct coaxial output, and up to hundreds of GHz when used with external waveguide source modules. Also FM and AM signal generators may be used.

The benefit of these different generators and others is they offer specific forms of power for targeted applications where one form of power has advantages over other forms. For example, when cutting and coagulating tissue, monopolar electricity typically can cut and coagulate through tissue more effectively than bipolar electrical energy. But monopolar energy requires the use of a grounding pad to avoid the arching of monopolar energy to unintentional areas. Hence, a grounding pad can be used with a monopolar application to affect tissue and prevent arching and subsequent electrical energy and burns to the patient with the monopolar energy. (The ground pad completes the circuit of the electrical energy through the patient.)

In contrast, bipolar electrical energy has a completed circuit in the device itself and therefore energy travels through and across the device, affecting tissue, but not arching through the body. With this approach, bipolar electrical energy can be very effective for creating lesions, sealing vessels and other applications involving targeted treatment of tissue. But, it tends to be less effective with cutting and coagulating through tissue as an alternative to a surgical knife because of the contained aspect of the bipolar electrical energy. Similarly, microwave energy may be used for certain types of ablation of tissue because of its unique tissue effect and bipolar energy may be used for other types of ablation. Other forms of energy, such as FM energy, may be used because the frequency does not excite certain collateral elements, such as nerve bundles.

A coblation generator can be used in the non-heat driven process of surgically disassociating soft tissue by using radiofrequency energy to excite the electrolytes in a conductive medium, such as saline solution, to create a precisely focused plasma field. Energized particles, or ions, in the plasma field can have sufficient energy to break, or dissociate, organic molecular bonds within soft tissue at relatively low temperatures, i.e., typically between 40° C. to 70° C. This enables coblation devices to volumetrically remove target tissue with minimal damage to surrounding tissue. Coblation can also provide hemostasis and tissue shrinkage capabilities. The amount of power delivered can be determined by intensity of the field and can be adjusted based on the local environmental condition.

Coblation may be used for temperature ranges typically up to 90° C.

An ultrasound generator is capable of generating acoustic waves having a frequency greater than approximately 20 kilohertz (20,000 hertz). The ultrasound waves may be conducted by the conductive material 302 to the tissue 200. Ultrasound can be absorbed by body tissues, especially ligaments, tendons, and fascia, or other matter.

Ultrasound devices can operate with frequencies typically from 20 KHz up to several GHz. Therapeutic ultrasound frequency used is 0.7 to 3.3 MHz. Ultrasound energy or TENS energy may speed up the healing process by increasing blood flow in the treated area, decrease pain from the reduction of swelling and edema, and gently massage the muscles tendons and/or ligaments in the treated area.

Ultrasound may also non-invasively or invasively to ablate tumors or other tissue. This can accomplished using a technique known as High Intensity Focused Ultrasound (HIFU), also called focused ultrasound surgery (FUS surgery). This procedure uses generally lower frequencies than medical diagnostic ultrasound (250-2000 kHz). Other general conditions which ultrasound may be used for treatment include such as examples as: ligament sprains, muscle strains, tendonitis, joint inflammation, plantar fasciitis, metatarsalgia, facet irritation, impingement syndrome, bursitis, rheumatoid arthritis, osteoarthritis, and scar tissue adhesion.

The device 600 also allows a medical practitioner to perform among others, cauterization of tissue, vessel sealing, tissue dissection and re-sectioning, tissue shaping, tissue cutting and coagulation, tissue ablation, and instrument heating, among others, all at the precise location that the practitioner is viewing. This at least partially addresses the problem of performing aspects of endoscopic surgery in the blind. It may also eliminate the need to exchange one device for another to apply energy to the tissue or matter or to deflect tissue or other matter or to engage in other manipulation while maintaining visualization.

More specific medical applications include, among others, application of energy to effect tissue in trauma cases, arthroscopic surgery, spine surgery, neurosurgery, shoulder surgery, lung tumor ablation, ablation of cancerous tissue with bladder cancer patients, cauterization or ablate uterine tissue for women's health issues (such as endometriosis). In these applications (and the other applications listed herein), the device can be used to contact tissue and then cauterize, ablate or shape the tissue (done with coblation energy for example in shoulder procedures), creating unique performance attributes by allowing the physician to see the change taking place to the tissue in real time through, for example, an optically clear material and coating.

To further elaborate on the medical applications, use of the device in diathermy applications is a useful area, whether achieved using short-wave radio frequency (range 1-100 MHz) or microwave energy (typically 915 MHz or 2.45 GHz). Diathermy used in surgery can comprise at least two types. Monopolar energy is where electrical current passes from one electrode near the tissue to be treated to the other fixed electrode elsewhere in the body. Usually this type of electrode is placed in a specific location on the body, such as contact with the buttocks or around the leg. Alternatively, bipolar energy can be used, where both electrodes are mounted in close proximity creating a closed electrical circuit on the device (in this case two separate conductive material portions 302 on the matter manipulator) and electrical current passes only through or on the tissue being treated. An advantage of bipolar electrosurgery is that it prevents the flow of current through other tissues of the body and focuses only on the tissue in contact or close proximity to the electrodes. This is useful in, for example, microsurgery, laparoscopic surgery, cardiac procedures and in other procedures, including those with patients with cardiac pacemakers and other devices and conditions not suitable for use with other forms of energy.

Electrocauterization is the process of modifying tissue using heat conduction from a metal probe heated by electric current. The procedure is used to stop bleeding from small vessels (larger vessels can be ligated) or for cutting through soft tissue. High frequency alternating current is used in electrocautery in unipolar or bipolar fashion. It can be continuous waveform (to cut tissue) or intermittent type (to coagulate tissue) or a combination to cut and coagulate. In unipolar type, the tissue to be coagulated/cut is to be contacted with small electrode, while the exit point of the circuit is large in surface area, as at the buttocks, to prevent electrical burns. Heat generated depends on size of contact area, power setting or frequency of current, duration of application, waveform. A constant waveform (generally) generates more heat than intermittent one because the frequency used in cutting the tissue is set higher than in coagulation mode. Bipolar electrocautery establishes circuit between two tips of and is used like forceps. It has the advantage of not disturbing other electrical rhythms of body (as in heart) and also acts to coagulate tissue by pressure.

As another option, the conductive layer 302 and device 600 may be used for thermal cautery in ranges of 50° C. through 100° C., or even in a range 50° C. through 70° C., or at lesser temperature if advisable, with the application of a range of power, appropriate to the application. Advantageously, the ability to visualize as forms of energy are applied through the device allows for the precise delivery of energy, including changing the level of energy and resulting temperature, using power settings appropriate to the specific application, applying energy over a longer period of time to broaden coverage, applying energy across multiple electrodes for multiple effects, and the ability to stop the process with more confidence that the tissue or other matter has been satisfactorily transformed. (This advantage of course applies to other applications of the device 600—real time visual monitoring of energy application allows for more precision application.)

This allows improved viewing and the ability to make repairs inside pipes, holding tanks, containers, hydraulic lines and other circumstances where visualization may otherwise be impaired, including when fluid is opaque, such as petroleum products, sewerage, food products, paint. Biologic drug manufacturing, pharmaceutical products and other applications would benefit from this innovation, eliminating the need to empty the pipes or containers (e.g., oil tanks) or open up the lines to inspect.

The size of the matter manipulator or the amount of flexibility can be scaled for specific applications, for example, displacing large volumes of fluid when examining large areas. The shape of the matter manipulator can be generally flat, convex (with varying levels of curvature), angled, sloped, stepped, or otherwise shaped for specific tasks. For example, the matter manipulator may be shaped as a square, or as an angular shape to displace opaque fluids in the corners of a tank to inspect the seams. Examination of joints, welds, seams for corrosion, pipes, flexible and non-flexible tubular members, or cracks, surface aberrations, and other points of inspection and repair could be performed in pipes, lines, tubes, tunnels, and other passages.

Matter manipulators with working channels will allow devices to be passed through a matter manipulator to make repairs using screws, adhesive patches, glues, chemicals, welding, soldering and other repair and modification applications. In embodiments, the matter manipulator can be formed from materials that resist acid, alkalinity, high heat, or viscosity of the fluid being displaced by the matter manipulator. In embodiments, the device could be a single-use disposable device or a reusable device.

Advantageously, implementations of the device 600 provide the ability to apply energy via the conductive material 302 in these varied non-medical applications. The energy provided to the viewed object may heat, alter or otherwise affect the object being manipulated by the matter manipulator.

Conductive Material Compositions

The conductive material 302 may have various compositions and be applied to the matter manipulator various ways. Examples of such compositions and applications are provided below for illustrative purposes and should not be considered limiting. For medical applications, the conductive material 302 preferable can withstand sterilization, such as by gamma irradiation, ethylene oxide, steam, or other forms of sterilization.

The electrically conductive/responsive coating can be applied in multiple configurations to create one or more electrodes. This electrode can be optically clear and of various thicknesses, including thickness of a half micron or less, and at much greater thicknesses, depending on the intended effect with tissue or other matter.

The conductive material can be at least partially transparent and can comprise for example, any member of the general class of materials known as transparent conductive oxides (TCOs), with titanium oxide (TiO₂) and aluminum-doped zinc oxide (AZO), being two examples. It could also involve applications of other conductive materials applied in a manner that permit visualization, such as silver and gold nanoparticles, and other conductive materials applied in a manner that allows for the conduction of energy and visualization.

A transparent conductive oxide may comprise transparent materials that possess bandgaps with energies corresponding to wavelengths which are shorter than the visible range of 380 nm to 750 nm. A film of a TCO can have a varying conductivity, for example, across points on the surface thereof. In one aspect, the film has no or substantially no pores, pinholes, and/or defects. In another aspect, the number and size of pores, pinholes, and/or defects in a layer do not adversely affect the performance of the layer in the device. The film thickness can range from less than 1 to about 3500 nm. In embodiments, different methods of fabrication and intended applications can lead to different thicknesses such as, for example, films about 10, 20, 30, 40, 50, 60, 70, 80, 100, 200, 300, 400, 500, 600, 700, 800, 900, 1000, 1300, and 1500 nm thick.

The transparent conductive film can be indium tin oxide, Al or Ga doped zinc oxide, Ta or Nb doped titanium oxide, F doped tin oxide, and their mixtures. The oxide layer can be formed by directly oxidizing an ultra-thin metal layer or by depositing an oxide. The TCO material can have polycrystalline, crystalline, or amorphous microstructures to affect the film properties, including for example, transmittance and conductivity, among other properties.

Biocompatible TCOs can also be used as the transparent conductive material. These include, for example, aluminum oxide (Al₂O₃), hydroxyapatite (HA), silicon dioxide (SiO₂) titanium carbide (TiC), titanium nitride (TiN), titanium dioxide (TiO₂), zirconium dioxide (ZrO₂). These materials may be n-doped with other metals such as aluminum, Al, copper, Cu, silver, Ag, gallium, Ga, magnesium, Mg, cadmium, Cd, indium, In, tin, Sn, scandium, Sc, yttrium, Y, cobalt, Co, manganese, Mn, chrome, Cr, and boron, B. p-Doping can be achieved with nitrogen, N, and phosphorus, P, among others.

TiO₂ can serve as a biocompatible material; it provides the possibility to coat substrates at temperatures ranging from room temperature to several hundreds of degrees centigrade. TiO₂ has multiple different polymorphic phases that can depend on the initial particle size, initial phase, dopant concentration, reaction atmosphere and annealing temperature. The TiO₂ films are commonly synthesized by many methods, including sol-gel, thermal spraying and physical vapor deposition.

Transparent conducting, aluminum doped zinc oxide thin films (AlₓZn_{y}O_{z}, ZnO:Al) contain a small amount (typically less than 5% by weight) of aluminum. The underlying substrate may have an influence on the grown structure and the opto-electronic properties of a film of the material. Even if the substrate is identical, the layer thickness (deposition time, position upon the substrate) itself influences the physical values of the deposited thin film.

A variation of the physical values from the grown thin films can also be reached by changing process parameters, as temperature or pressure, or by additions to the process gas, as oxygen or hydrogen. Commonly, zinc oxides are n-doped with aluminum. Alternatively, n-doping can be done with metals such as copper, Cu, silver, Ag, gallium, Ga, magnesium, Mg, cadmium, Cd, indium, In, tin, Sn, scandium, Sc, yttrium, Y, cobalt, Co, manganese, Mn, chrome, Cr, and boron, B. The p-Doping of ZnO can be achieved with nitrogen, N, and phosphorus, P.

Additionally, the incorporation of sub-wavelength metallic nanostructures in TCO can result in changes to the wavelength where the TCO becomes transparent. Embedded particles articles can also be used to control absorption and scattering at desired wavelengths. Other optical effects of the material can be influenced as well including absorption, scattering, light trapping or detrapping, filtering, light induced heating and others. The morphology of the particles (including size, shape, density, uniformity, conformity, separation, placement and random or periodic distribution) can be used to engineer these effects.

For optically transparent applications, the substrate of the electrode of the invention can be of any suitable material on which the transparent electrode structure of this invention is applied. This can include another conductive material or a dielectric material. In one illustrative example, the matter contacting portions of the matter manipulator serves as the substrate. Other substrates include, among others, glass, a semiconductor, an inorganic crystal, a rigid or flexible plastic material. Illustrative examples are silica (SiO₂), borosilicate (BK7), silicon (Si), lithium niobate (LiNbO₃), polyethylene naphthalate (PEN), polyethelene terephthalate (PET), among others.

Organic materials can also serve as the conductive material. These include carbon nanotube networks and graphene, which can be fabricated to be highly transparent to infrared light, along with networks of polymers such as poly(3,4-ethylenedioxythiophene) and its derivatives.

Polymers can also serve as the conductive material. For example, conductive polymers such as derivatives of polyacetylene, polyaniline, polypyrrole or polythiophenes. poly(3,4-ethylenedioxythiophene) (PEDOT), and PEDOT: poly(styrene sulfonate) PSS. Additionally, Poly(4,4-dioctylcyclopentadithiophene) doped with iodine or 2,3-dichloro-5,6-dicyano-1,4-benzoquinone (DDQ) can be used. Other polymers with n or p type dopants can also be used.

Conductive material films can be deposited on a substrate through various deposition methods, including metal organic chemical vapor deposition (MOCVD), metal organic molecular beam deposition (MOMBD), spray pyrolysis, and pulsed laser deposition, dip coating, painting, gluing or other applications suitable for appropriately adhering the conductive materials to the given substrate for the particular application. Fabrication techniques of TCOs include magnetron sputtering of the film, sol gel technology, electro deposition, vapor phase deposition, magnetron DC sputtering, magnetron RF sputtering or a combination of both the sputter deposition methods, ultrasonic delivery and welding. Moreover, high quality deposition methods using thermal plasmas, (low pressure (LP), metal organic (MO), plasma enhanced (PE)) chemical vapor deposition (CVD), electron beam evaporation, pulsed laser deposition and atomic layer deposition (ALD) can be applied, among others.

A thin film, such as ALD, only a few nanometers thick can be flexible and thus less prone to cracking and formation and spreading of detrimental particles inside the human body or insider the given non-medical inspection site. Also, low and high protein binding affinity coatings can be deposited by ALD. They are especially useful in diagnostics and in the preparative field, as well as for surface coatings that resist bacterial growth.

Pre and post deposition processing such as processing with an oxygen plasma and thermal treatment can be combined to obtain improved conductive material characteristics. The oxygen plasma might be preferable for when the substrate, or conductive material would be affected by the high temperatures. The conductive material film can have a wide range of material properties depending on variations in process parameters. For example, varying the process parameters can result in a wide range of conductivity properties and morphology of the film.

A number of aspects of the systems, devices and methods have been described. Nevertheless, it will be understood that various modifications may be made without departing from the spirit and scope of the disclosure. Accordingly, other aspects are within the scope of the following claims.

That which is claimed:

1. A device comprising:
a tissue manipulator;
a conductive coating disposed on at least a portion of the tissue manipulator, the conductive coating configured for energy conduction;
at least one connector area capable of supplying energy to the conductive coating.

2. The device of claim 1, wherein the conductive coating is at least partially optically transparent.

3. The device of claim 2, wherein the conductive coating includes a conductive oxide.

4. The device of claim 3, wherein the conductive oxide is selected from the group consisting of: a titanium conductive oxide and an aluminum conductive oxide.

5. The device of claim 1, wherein the connector area is configured for connection to a power source.

6. The device of claim 5, further comprising the power source.

7. The device of claim 6, wherein the power source is selected from the group consisting of: an electrical energy generator, an electrosurgical generator, a coblation generator, an ultrasound generator, an argon gas generator, and a plasma generator.

8. The device of claim 1, wherein the tissue manipulator is a fastener.

9. The device of claim 8, wherein the fastener is a staple.

10. The device of claim 8, wherein the portion of the tissue manipulator includes a tissue-adjacent surface of the fastener.

11. The device of claim 10, wherein the conductive coating is configured to generate sufficient energy to alter tissue.

12. The device of claim 10, wherein sufficient energy to alter tissue is sufficient energy for one of a group consisting of: ablate, cauterize, shape, seal, dissect, resect, debride, cut, declot and coagulate tissue.

13. The device of claim 1, wherein an area of the conductive coating and an area the tissue manipulator are at least partially optically transparent.

14. The device of claim 13, wherein optically transparent areas are overlapping and positionable on tissue being manipulated and energized.

15. The device of claim 1, wherein the conductive coating is configured to convert electrical energy to thermal energy.

16. The device of claim 1, wherein the tissue manipulator is configured to transmit a force onto tissue.

17. The device of claim 16, wherein the tissue manipulator includes one of a group consisting of a snare, a suture, a fastener, a staple, a clip, a clamp, an anastomosis device, an anchoring device, a ligature, scissors, jaws and a knife.

18. The device of claim 16, wherein the tissue manipulator is at least partially constructed of a biocompatible material.

19. The device of claim 18, wherein the biocompatible material is configured to provide adherence of the conductive coating.

20. The device of claim 1, wherein the conductive coating has a thickness of half a micron or less.

21. The device of claim 1, wherein the tissue manipulator is a snare and wherein the energy supplied is bipolar energy.

22. A method comprising:
contacting at least a portion of a tissue with a manipulator;
applying energy to a coating on the manipulator; and
altering the portion of the tissue by conducting the energy onto the portion of the tissue using the coating on the manipulator.

23. The method of claim 22, wherein altering the portion of the tissue includes heating the portion of the tissue.

24. The method of claim 22, wherein applying energy includes applying a bipolar electrical energy.

25. The method of claim 24, wherein altering the portion of the tissue includes cauterizing the portion of the tissue.

26. The method of claim 22, wherein contacting the tissue includes stapling the tissue using a staple as the manipulator.

27. The method of claim 22, wherein altering the portion of the tissue includes ablating the portion of the tissue.

28. The method of claim 22, further comprising simultaneously viewing the portion of the tissue while altering the portion of the tissue.

29. The method of claim 22, wherein altering the portion of the tissue includes one of a group consisting of: ablating, cauterizing, shaping, sealing, dissecting, resecting, cutting and coagulating the portion of the tissue.

30. The method of claim 22, wherein contacting the tissue includes cutting the tissue using a knife as the manipulator and wherein altering the tissue includes cauterizing the tissue by conducting electricity using the coating on the knife.

31. The device of claim 1, wherein the tissue manipulator is a tissue closure.

32. The device of claim 31, wherein the tissue closure includes at least one of a group consisting of sutures, staples, fasteners, clips, clamps, anastomosis device and glues.

33. The device of claim 31, wherein the tissue closure includes a glue configured to be activated by energy.

34. The device of claim 1, wherein the tissue manipulator is a tissue support.

35. The device of claim 34, wherein the tissue support includes one of a group consisting of a hernia mesh, a vaginal mesh and a uterine repair device.

36. The device of claim 34, wherein the tissue support is a synthetic mesh.

37. The device of claim 34, wherein the tissue support is a biologic mesh.

38. The device of claim 1, wherein the tissue manipulator is a cage.

39. The device of claim 1, wherein the tissue manipulator is a stent.

40. The device of claim 1, wherein the tissue manipulator is a clip.

41. The device of claim 1, wherein the tissue manipulator is an anchoring device.

42. The device of claim 1, where the matter manipulator is bioabsorbable.

43. The device of claim 1, wherein the matter manipulator is an energy probe.

44. The device of claim 43, wherein the conductive coating is hydrophobic.

45. The device of claim 43, wherein the conductive coating is hydrophilic.

46. The device of claim 43, wherein the conductive coating is super-hydrophobic.

47. The device of claim 1, wherein the tissue manipulator is a guide wire or a catheter.

48. The device of claim 47, wherein the tissue manipulator is configured for use in a lumen.

49. The device of claim 1, wherein the coating is configured to further be hydrophilic.

50. The device of claim 1, wherein the coating is configured to further be hydrophobic.

51. The device of claim 1, wherein the coating is configured to be super-hydrophobic.

52. The method of claim 22, wherein alternating the portion of the tissue includes sealing the tissue by applying energy to the coating on a knife or a clamp.
